(19) United States
(12) Patent Application Publication
(10) Pub. No.: US 2018/0344443 A1
(43) Pub. Date: Dec. 6, 2018

(54) INJECTING GUN FOR THE TREATMENT OF ANIMALS WITH DRIVEN BUILT-IN COUNTER

(71) Applicant: DIGIRODEO S.A., Buenos Aires (AR)

(72) Inventors: Carlos Ignacio Iturriaga, Buenos Aires (AR); Fernando Oscar Diaz Offeney, Buenos Aires (AR); Nicolas Alejandro Reyes Viserie, Buenos Aires (AR); Leopoldo Simini, Buenos Aires (AR)

(73) Assignee: DIGIRODEO S.A., Buenos Aires (AR)

(21) Appl. No.: 15/989,789

(22) Filed: May 25, 2018

(30) Foreign Application Priority Data
Jun. 1, 2017 (AR) P170101488

(51) Int. Cl. A61D 7/00; A61M 5/31

(52) U.S. Cl. CPC A61D 7/00; A61M 5/31; A61M 2005/3126; A61M 2205/3584; A61M 2205/502; A61M 2250/00; A61M 2205/582; A61M 2205/583; A61M 2205/586; A61M 2205/587; A61M 2205/8212; A61M 2205/581

(57) ABSTRACT

An injecting gun with driven built-in counter, having a loading tube with a support for a needle at its distal end and a sliding plunger attached to a rod with transversal slots ending in a handle; a trigger associated with a cam that, when triggered, pushes the rod through the slots where said cam is settled, and a body including therein an electronic card constituted by a CPU connected to: a) GPIO modules connected with: i) a plurality of tactile switches, ii) a balance switch, iii) a OLED display, iv) a keyboard with associated LEDs, v) a buzzer, and vi) a speaker; y b) a USB module connected with: vii) a micro USB plug; wherein, said CPU is linked to a memory; tactile switches are associated to the trigger; and an energy source with energy manager is connected to said CPU and modules providing the energy needed for functioning.

### CROSS-REFERENCE TO RELATED APPLICATIONS

This application claims the benefit of priority to Argentina International Application Serial No. P170101488, filed Jun. 1, 2017, the contents of which are hereby incorporated by reference.

### FIELD OF THE INVENTION

The present invention belongs to the field of veterinary accessories for administering injections, more preferably, it refers to those devices that allow counting doses effectively administered, by category of predetermined treated animal, even more preferably, it is related to gun-type syringes that may process, store and transfer information on a treatment.

### DESCRIPTION OF PRIOR ART

Typically, injections in animals are given with gun syringes where each activation of the trigger corresponds to a dose delivered. These doses are measured in cubic centimeters, and generally, the amount given may be regulated within the range between 1 cm³ y 5 cm³ depending on the vaccine or the drug being administered.

In several situations, the amount of administrations is calculated approximately as per the number of vials or bottles of used vaccine or veterinary product or, simply, they are not counted. This is how, typically, once the vaccinated cattle passes through the tube, the work is finished with no registration at al.

Most of syringes currently on the market do not have application counter, as they neither have administered effective dose identifying device. This way, one may know the exact number of cattle to be vaccinated, but there is no precise information on how many applications were really performed by the end of work.

Likewise, there are situations where there is no exact knowledge on the number of animals present in a troop to be treated and the vaccination work is used to count them manually.

Sometimes they are passed from a corral to another one to count them as they pass through a gate or tube, but this management is against the welfare of the animals since it stresses them when making unnecessary non-routine movements.

In order to count the animals of the rodeos at the same time they are vaccinated, a technique was implemented which allows performing both tasks simultaneously without impeding each other. There are devices that allow separating the vaccinator from the work of counting animals, which is a very important complication in huge troops.

These devices are an invaluable tool that allows establishing a control on vaccinated animals, which are submitted to official vaccination plans, such as foot and mouth disease or brucellosis. Thus, the official vaccinator will be able, for example, to verify that the informed animals to be vaccinated match the administered doses.

In order to solve this inconvenience, some devices have been proposed that may be coupled to a syringe which can accurately count the number of doses administered thus improving the performance of said element by facilitating, at the same time, the work of vaccinating personnel.

Particularly, patent AR080390B1 refers to a device that, when being coupled to a vaccination syringe, digitally counts the number of administrations performed. A button is pressed when triggering said syringe and a signal is sent to the digital counter, which sums up by 1 (one) unit with a range from 0 to 99999. The main advantage of this complementary product is the accuracy and the control achieved on the quantitative number of doses administered.

On the other hand, document AR092254A4 refers to a digital counting device to be applied on gun syringes to inject animals, driven at the handle comprising two bodies electrically connected to each other, wherein the first body is formed by two main housings linked by their edges that limit a cylindrical free space de forma and have an electronic card inside formed by: a circuit formed by a counter, a screen for data acquisition from outside connected to the built-in circuit, a reset switch connected to the built-in circuit accessible from outside, a power source connected to the built-in circuit, and a sound signal generator (beeper) connected to the power source and to the built-in circuit; and the second body is formed by: two secondary housings linked through their edges comprising inside: a two-cannel button for opening/closing of the counter/beeper circuit; wherein both bodies are linked through a connection cable connecting the built-in circuit with the button with a switch between the counter built-in circuit and the button.

However, in spite of the improvement reached with this device, there is still a need for a vaccination device that allows effectively counting the administered doses, such as vaccinations and disease treatments. This accounting of effective applications is extremely important in treatments on animals.

Therefore, it is an object of this invention to provide for an injecting gun capable of recording the effectively administered doses, by animal category or a lot predetermined by the operator, so that false positives can easily be discarded when the gun is triggered to eliminate an air bubble, at the same time facilitating the work of the staff in charge of this task. It is also convenient to be able to process and save the obtained information by subsequently sending it to another device for evaluation and storage.

### SUMMARY OF THE INVENTION

Therefore, it is an object of the present invention to provide an injecting gun for the treatment of animals with a built-in driven counter, said injecting gun, comprising:

a load tube comprising a support for a needle on its distal end and a sliding load plunger attached to a rod with transversal slots ending in a handle;

a trigger associated to a cam that, when actuated, pushes the rod by means of the slots on which the cam rests, being its path regulated by a selecting wheel pivotally mounted on an axis linked to an internal fixed support to which said trigger is separated by means of a spring; and

a body formed by two main housings linked on their edges that form an internal free space, said body sustaining said load tube showing its distal end while the housings substantially form the handle to the front of which and beneath the tube is the trigger;

a CPU (Central Processing Unit) which is connected to:
a) GPIO modules (General Purpose Input/Output) modules connected to:
   i) a plurality of tactile switches (microswitches),
   ii) a rolling switch,
   iii) OLED screen (Organic Light-Emitting Diode),
   iv) a keyboard with associated LEDs (Light Emitting Diode),
   v) a vibrator, and
   vi) a speaker; and
b) one USB (Universal Serial Bus) module connected to:
   vii) one micro USB plug (micro USB);

wherein said CPU is linked with a memory;

wherein the tactile switches are associated to the trigger; and

wherein a power source with energy manager is connected to said CPU and modules supplying the energy needed for its functioning.

Preferably, said CPU is a built-in component and it is selected from Cortex-M0, Cortex-M0+, Cortex-M3, Cortex-M4, Cortex-M7, Cortex-M23, and Cortex-M33

More preferably, said CPU is a built-in component Cortex-M3 120 MHz.

Preferably, the plurality of tactile switches (microswitches) are sequentially activated by actuating the trigger of the injecting gun, assuring that the application of the injection was made correctly, validating the application and saving it in the memory.

More preferably, the plurality of tactile switches comprise 5 tactile switches.

In an also preferred embodiment, the rolling switch deactivates the plurality of tactile switches when the injecting needle of the injecting gun is in vertical position, thus blocking the counting.

In a more preferred embodiment, the vertical position of the injecting needle of the injecting gun is between 75° and 90° as regards the horizontal axis.

Preferably, the LEDs (Light Emitting Diode) associated to the keyboard, the vibrator and the speaker provide the visible, tactile and sound, respectively, during the injecting gun operation.

Also preferably, the USB module (Universal Serial Bus) connected to the CPU allows for the transfer of information from the memory connected to the CPU to an external device wirelessly connected to the micro USB plug, or vice versa.

Also preferably, said memory is an internal memory inside the CPU, an external SD memory card (SD card, Secure Digital card), or a combination of both.

Even more preferably, the external device is a cell phone, a Tablet, a Notebook, or a personal computer (PC).

Preferably, the energy source is at least a 9 V rechargeable battery selected from NiCd (Nickel-Cadmium), NiMH (Nickel-Metal Hydride), Li-Ion (Lithium-Ion) and LiPo (Lithium Polymer), or a non-rechargeable battery selected from alkaline, ZnC (Zinc-Carbon) and Li (Lithium).

More preferably, said energy manager comprises a SMPS source (Switching Mode Power Supply) that transforms the output current of the battery into a 3.9 V/300 mA current, a lineal parallel source transforming the output current of the battery into an auxiliary 3.3 V/150 mA current, and a linear source in series with the SMPS source that transforms the current exiting said source into a 3.3 V/250 mA current.

Also, the injecting gun comprises a UART module (Universal Asynchronous Receiver/Transmitter) connected to the CPU, this module being a Bluetooth module connected to the CPU that allows wirelessly transferring information from the memory connected to the CPU to an external device.

### DESCRIPTION OF THE FIGURES

FIG. 1 shows a perspective lateral top view of a preferred embodiment of an injecting gun according with the present invention.

FIG. 2 shows a perspective lateral top view of a preferred embodiment of the injecting gun of FIG. 1.

FIG. 3 shows a block diagram of a preferred embodiment of the electronic circuit of the injecting gun in agreement with the present invention.

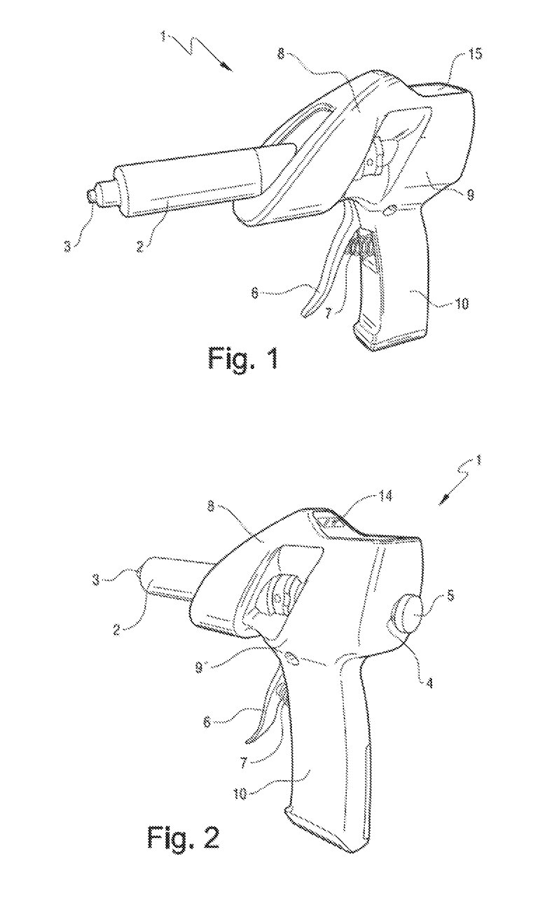

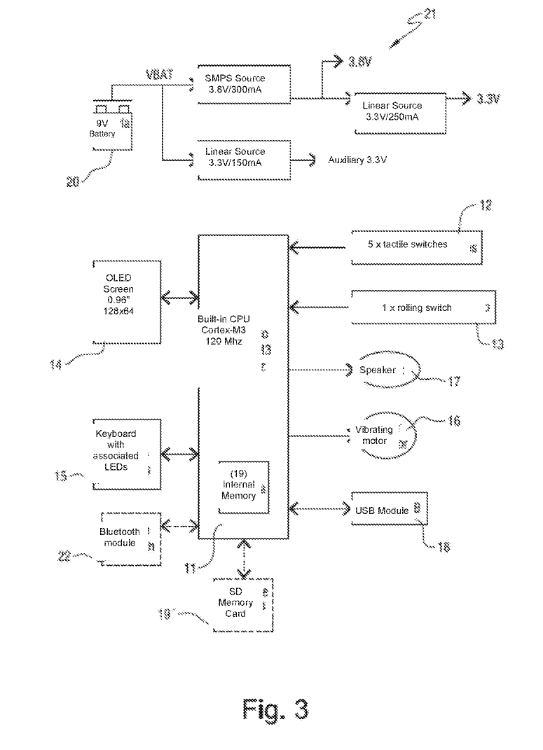

### DETAILED DESCRIPTION OF THE INVENTION

Therefore, it is an object of the present invention to provide an injecting gun (1) that allows recording, by animal category or by pre-selected lot of animals, the effectively administered dose in vaccinations or disease treatments. This recording is of extreme importance for the follow up of treatments performed to animals and the health thereof.

In a preferred embodiment of the present invention, the injecting gun (1) for the treatment of animals with driven built-in counter comprises:

a load tube (2) comprising a support (3) for a needle on its distal end and a sliding load plunger attached to a rod (4) with transversal slots ending in a handle (5);

a trigger (6) associated to a cam that, when actuated, pushes the rod (4) by means of the slots on which the cam rests, being its path regulated by a selecting wheel pivotally mounted on an axis linked to an internal fixed support to which said trigger (6) is separated by means of a spring (7); and

a body (8) formed by two main housings (9, 9′) linked on their edges that form an internal free space, said body (8) sustaining said load tube (2) showing its distal end while the housings (9, 9′) substantially form the handle (10) to the front of which and beneath the load tube (2) is the trigger (6);

wherein said internal free space includes an electronic card made of:

a CPU (Central Processing Unit) (11) which is connected to:
a) GPIO modules (General Purpose Input/Output) modules connected to:
   i) a plurality of tactile switches (microswitches) (12),
   ii) a rolling switch (13),
   iii) a OLED screen (Organic Light-Emitting Diode) (14),
   iv) a keyboard with associated LEDs (Light Emitting Diode) (15),
   v) a vibrator (16), and
   vi) a speaker (17); and
b) a USB (Universal Serial Bus) module (18) connected to:
   vii) one micro USB plug (micro USB);

wherein said CPU (11) is linked to a memory (19, 19′);

wherein tactile switches are associated with the trigger (6); and

wherein an energy source (20) with energy manager (21) is connected to said CPU (11) and modules providing the energy needed for operation.

Basically, the loading of the injecting gun (1) is performed once the needle is positioned on the end of the load tube (2) by punching the bottle through an elastic means, normally the cap or the stopper thereof, where the vaccine or the treatment solution is located, and pushing the syringe plunger backwards by means of the handle (5). This makes the product is drawn out entering the load tube (2).

Normally, the load tube (2) has about 50 cm³ in volume. The load duration will depend on the dose to be inoculated to each animal. For example, the average volume normally used in vaccines is 5 cm³ per animal, and in this case up to 10 animals may be vaccinated, then the load tube (2) of the injecting gun (1) is loaded again. There are vaccines that need 2 cm³ per animal, and in this case, up to 25 animals per load may be charged.

The injecting gun (1) has a selecting gun that limits the path of the plunger in each stroke of the trigger (6). This allows injecting, for example, 1, 2, 3, 4 or 5 cm³. In the case more than volume should be injected per animal, the application is repeated. For example, if the volume to be injected is 8 cm³, two consecutive applications of 4 cm³ each are performed.

In each application or stroke of the trigger (6) of the injecting gun (1), the plunger of the load tube (2) is pushed by a rod (4) with slots associated thereto by a sting linked to the trigger (6) that fits on the slots of the rod (4), said stroke being regulated by the selecting wheel that limits the injected volume.

After each load of the product to be administered, the residual air left in the end of the load tube (2) and the needle should be eliminated.

Preferably, the CPU (11) of the injecting gun (1) according to the present invention may be a built-in component and it is selected from Cortex-M0, Cortex-M0+, Cortex-M3, Cortex-M4, Cortex-M7, Cortex-M23, and Cortex-M33 Particularly, the built-in circuit identified as Cortex-M3 120 MHz may be used as the CPU (11).

Eventually, any other built-in element may be used which meets the same expectations.

The injecting gun (1) comprises a plurality of tactile switches (microswitches) (12) that are sequentially activated by actuating the trigger (6) of the injecting gun (1), assuring that the application of the injection with the vaccine or treatment is truly concreted, validating the administration and saving it into a memory (19, 19′).

In a particular embodiment the plurality of tactile switches (12) of the injecting gun (1) of the present invention comprises 5 tactile switches (12).

By activating the trigger (6) of the injecting gun (1), it presses the tactile switches (12) along the trajectory, said switches being in a series, i.e., they are one after the other.

The last switch is activated by performing an application and validates the reader reading, confirming that the type of animal or lot previously identified by the operator has been vaccinated or treated therefore saving the data into the memory (19, 19′).

In a preferred embodiment, the injecting gun (1) in agreement with the present invention comprises a rolling switch (13), which deactivates tactile switches (12) when the injecting needle of injecting gun (1) is in vertical position, this way blocking the counting of false positives during the vaccination or treatment proceeding.

Also preferably, the above mentioned vertical position of the injecting needle of the injecting gun (1) is located between 75° and 90° regarding the horizontal axis.

Through the OLED screen (14) it is possible to follow the counting progress during the vaccination or treatment operation.

The injecting gun (1) in agreement with the present invention comprises LEDs (Light Emitting Diode) associated to the keyboard (15), a vibrator (16) and a speaker (17), which respectively provide visible, tactile and sound signals during the injecting gun (1) operation. This way the user has information during the vaccination or treatment proceeding rapidly indicating him the situation.

The injecting gun (1) in agreement with the present invention comprises a memory (19, 19′) which stores data on the vaccination or treatment proceeding while this is carried out.

Preferably, the memory (19) may be internal and built-in in the CPU (11), it may be a SD memory card (SD card, Secure Digital card) (19′) which is external to the CPU (11), or also it may be a combination of both types of memory (19, 19′) as the requirement may be.

All the information on the performed vaccination or treatment is stored in the memory (19, 19′) of the injecting gun (1) and may be transferred by means of a USB port (18) to another auxiliary device in possession of the operator carrying out the vaccination or treatment.

In fact, each dose of vaccine or medication, such as for example an antibiotic or anti-parasitic used in the treatment of an animal is identified by the type or by the effective counting, thus eliminating false positives that may occur when an air bubble is expelled from the syringe. After that, data is stored by the previously selected category of animal or lot of animals and they are finally transferred to another external device by means of said USB connection (18).

The injecting gun (1) in agreement with the present invention then comprises, a USB module (Universal Serial Bus) (18) connected to the CPU (11) that allows transferring the information stored in the memory from the memory (19, 19′) connected to the CPU (11) to an external device wirelessly connected to the micro USB plug (micro USB) or vice versa.

This way, the data on the treatment is transferred to the external device where it may be stored and processed, and from the external device, for example, software updates may be transferred.

Preferably, the information is transferred from the injecting gun (1) to an external device that may be a cell phone, a Tablet, a Notebook, or a personal computer (PC), there a storage of data may be performed along with its processing by means of a computer program installed in said devices.

The injecting gun (1) in agreement with the present invention has an energy source (20). The energy source (20) that may preferably be used is at least one rechargeable 9 V battery selected from NiCd (Nickel-Cadmium), NiMH (Nickel-Metal Hydride), Li-Ion (Lithium-Ion) and LiPo (Lithium Polymer), or a non-rechargeable battery selected from alkaline, ZnC (Zinc-Carbon) and Li (Lithium).

Likewise, the injecting gun (1) of the present invention preferably has an energy manager (21) comprising a SMPS source (Switching Mode Power Supply) that transforms the output current of the battery to a 3.9 V/300 mA current, a linear parallel source that transforms the output current of the battery into an auxiliary 3.3 V/150 mA current, and a linear source in series with the SMPS source that transforms the output current of said source into a 3.3 V/250 mA current.

This say, there are three outputs to different voltages and amperages that are alternately complementary to supply the device with energy.

Normally, the accessory circuitry of the modules connected to the CPU (11) take the necessary energy from the same CPU (11) to function adequately.

In a preferred embodiment of the present invention, the injecting gun (1) additionally comprises a UART module (Universal Asynchronous Receiver/Transmitter) connected to the CPU (11), this module being a Bluetooth module (22) connected to the CPU (11) that allows wirelessly transferring information from the memory (19, 19′) connected to the CPU (11) to an external device having this technology such as any of the ones mentioned above, that is a cell phone, a Tablet, a Notebook, or a personal computer (PC), or any other similar portable device.

Likewise, the CPU (11) has a computer program installed with instructions that allow the injecting gun (1) providing the user with all the supplies and facilities needed to perform the tasks related with the identification, by type of animal or lot of animals, and the type of treatment of the animals as described herein.

The objects proposed and the benefits achieved were reached by the realization of an injecting gun (1) for the treatment of animals, with effective dose counting which is activated by means of the mechanical actuation of the trigger (6) thereof when administering a dose, without counting the expulsions of air bubbles or false counting.

The objective of the injecting gun (1) of the present invention is to increase the service and professionalism quality standards of the livestock sector, allowing the possibility of having a device that, with previous determination of the type of animal or lot of animals to be treated by the operator, such as for example, bulls, steers, heifers, etc., this information is associated with an effective counting and typing of performed administrations by means of the injecting gun (1) according to the present invention.

Having described the nature of the present invention and the way it is practiced, it is stated that the following is claimed as exclusive property and invention of the applicants:

1. An injecting gun for the treatment of animals with a built-in driven counter, said injecting gun characterized in that it comprises:
a load tube comprising a support for a needle on its distal end and a sliding load plunger attached to a rod with transversal slots ending in a handle;
a trigger associated to a cam that, when actuated, pushes the rod by means of the slots on which the cam rests, being its path regulated by a selecting wheel pivotally mounted on an axis linked to an internal fixed support to which said trigger is separated by means of a spring; and
a body formed by two main housings linked on their edges that form an internal free space, said body sustaining said load tube showing its distal end while the housings substantially form the handle to the front of which and beneath the tube is the trigger;
wherein said internal free space includes an electronic card made of:
a CPU (Central Processing Unit) which is connected to:
a) GPIO modules (General Purpose Input/Output) modules connected to:
i) a plurality of tactile switches (microswitches),
ii) a rolling switch,
iii) OLED screen (Organic Light-Emitting Diode),
iv) a keyboard with associated LEDs (Light Emitting Diode),
v) a vibrator, and
vi) a speaker; and
b) one USB (Universal Serial Bus) module connected to:
vii) one micro USB plug (micro USB);
wherein said CPU is linked with a memory;
wherein the tactile switches are associated to the trigger; and
wherein a power source with energy manager is connected to said CPU and modules supplying the energy needed for its functioning.

2. The injecting gun according to claim 1, characterized in that said CPU is a built-in component and it is selected from Cortex-M0, Codex-M0+, Cortex-M3, Cortex-M4, Cortex-M7, Cortex-M23, and Cortex-M33.

3. The injecting gun according to claim 2, characterized in that said CPU is a built-in Cortex-M4 120 MHz component.

4. The injecting gun according to claim 1, characterized in that the plurality of tactile switches (microswitches) is sequentially activated when triggering the injecting gun assuring that the administration of the injection was performed correctly, validating the administration and saving it into the memory

5. The injecting gun according to claim 4, characterized in that the plurality of tactile switches comprises 5 tactile switches.

6. The injecting gun according to claim 1, characterized in that the rolling switch deactivates tactile switches when the injecting needle of the injecting gun is in vertical position blocking the counting.

7. The injecting gun according to claim 6, characterized in that the vertical position of the injecting needle of the injecting gun is between 75° y 90° regarding the horizontal axis.

8. The injecting gun according to claim 1, characterized in that the LEDs (Light Emitting Diode) associated to the keyboard, the vibrator and the speaker provide visible, tactile and sound signals, respectively, during the injecting gun operation.

9. The injecting gun according to claim 1, characterized in that the USB module (Universal Serial Bus) connected to the CPU allows transferring information from the memory connected to the CPU to an external device wirelessly connected to the micro USB plug (micro USB) or vice versa.

10. The injecting gun according to claim 9, characterized in that said memory is internal, built-in in the CPU, and it is a SD memory card (SD card, Secure Digital card).

11. The injecting gun according to claim 9, characterized in that the external device is a cell phone, a Tablet, a Notebook, or a personal computer (PC).

12. The injecting gun according to claim 1, characterized in that the energy source is at least one rechargeable 9 V battery selected from NiCd (Nickel-Cadmium), NiMH (Nickel-Metal Hydride), Li-Ion (Lithium-Ion) and LiPo (Lithium Polymer), or a non-rechargeable battery selected from alkaline, ZnC (Zinc-Carbon) and Li (Lithium).

13. The injecting gun according to claim 1, characterized in that said energy manager comprises a SMPS source (Switching Mode Power Supply) that transforms the output current of the battery into a 3.9 V/300 mA current, a lineal parallel source transforming the output current of the battery into an auxiliary 3.3 V/150 mA current, and a linear source in series with the SMPS source that transforms the current exiting said source into a 3.3 V/250 mA current.

14. The injecting gun according to claim 1, characterized in that additionally it comprises a UART module (Universal Asynchronous Receiver/Transmitter) connected to the CPU, this module being a Bluetooth module connected to the CPU that allows wirelessly transferring information from the memory connected to the CPU to an external device.
